(19) United States
(12) Patent Application Publication
(10) Pub. No.: US 2013/0150864 A1
(43) Pub. Date: Jun. 13, 2013

(54) SURGICAL INSTRUMENT AND METHOD

(71) Applicants: Gregory C. Marik, Collierville, TN (US); Siddiqui Danish, Northbrook, IL (US); Trevor Seck, Memphis, TN (US)

(72) Inventors: Gregory C. Marik, Collierville, TN (US); Siddiqui Danish, Northbrook, IL (US); Trevor Seck, Memphis, TN (US)

(73) Assignee: Warsaw Orthopedic, Inc., Warsaw, IN (US)

(21) Appl. No.: 13/323,039

(22) Filed: Dec. 12, 2011

(51) Int. Cl. A61B 17/56

(52) U.S. Cl. 606/104

(57) ABSTRACT

A surgical instrument includes a first member extending along a longitudinal axis having a first end configured to mate with a head of a threaded member, the first end having a first configuration such that the first end loosely mates with the head of the threaded member and a second configuration such that the first end tightly mates with the head of the threaded member. The surgical instrument further includes a second member configured to expand the head from the first configuration to the second configuration. Methods of use are disclosed.

### TECHNICAL FIELD

The present disclosure generally relates to medical devices for the treatment of musculoskeletal disorders, and more particularly to a surgical instrument and method, which include a driver for driving a threaded member.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Fusion and fixation treatment may employ implants such as interbody fusion devices to achieve arthrodesis. Implants may also be used in other treatments such as arthroplasty. Surgical treatments employing minimally invasive techniques may use threaded members such as screws and bolts for fixing an implant in a specific configuration or securing an implant to a surgical site. This disclosure describes an improvement over these prior art technologies.

### SUMMARY OF THE INVENTION

Accordingly, a surgical instrument and related methods are provided for treating musculoskeletal disorders. It is contemplated that the surgical instrument and methods disclosed include an expandable locking hex driver.

In one particular embodiment, in accordance with the principles of the present disclosure, a surgical instrument is provided. The surgical instrument includes a first member extending along a longitudinal axis having a first end configured to mate with a head of a threaded member. The first end having a first configuration such that the first end loosely mates with the head of said threaded member and a second configuration such that the first end tightly mates with the head of said threaded member. The surgical instrument further includes a second member configured to expand the head from the first configuration to the second configuration.

In one embodiment, the surgical instrument includes a first member extending along a longitudinal axis having a first end configured to mate with a head of a threaded member. The first end having a first configuration such that the first end loosely mates with the head of the threaded member and a second configuration such that the first end tightly mates with the head of the threaded member. The surgical instrument further includes a locking pin insertable into a bore extending through the first member along the longitudinal axis. The locking pin is configured to expand the head from the first configuration to the second configuration. The locking pin is threadingly engageable with the first member to advance the locking pin through at least a portion of the bore. The surgical instrument still further includes a handle positioned at a second end opposite the first end of the first member. The handle defines a bore aligned with the bore of the first member. The locking pin is insertable through the bore of the handle to continue through the bore of the first member.

In one embodiment, a method of providing a threaded member during a surgical procedure is provided. The method includes providing a surgical instrument having a first configuration wherein a head of the surgical instrument loosely mates with a head of the threaded member and a second configuration wherein the head of the surgical instrument tightly mates with the head of the threaded member. The method also includes providing the threaded member. The method further includes mating the threaded member with the head of the surgical instrument. The method still further includes expanding the surgical instrument from the first configuration to the second configuration to provide a holding force to the threaded member. The mother further includes driving the threaded member into a patient. The method still further includes collapsing the surgical instrument from the second configuration to the first configuration. The method also includes removing the surgical instrument from the threaded member. The method includes removing the surgical instrument from the patient.

### BRIEF DESCRIPTION OF THE DRAWINGS

The present disclosure will become more readily apparent from the specific description accompanied by the following drawings, in which:

FIG. 1 is a perspective view of one embodiment of a surgical instrument in accordance with the principles of the present disclosure;

FIG. 2 is an exploded, perspective view of the surgical instrument shown in FIG. 1 with parts separated;

FIG. 3 is an enlarged, cutaway side view of a proximal end of a locking pin of the surgical instrument shown in FIG. 1;

FIG. 4 is a enlarged, cutaway side view of a proximal end of a first member of the surgical instrument shown in FIG. 1;

FIG. 5 is an enlarged, cutaway cross-section view of the proximal end of the first member of the surgical instrument shown in FIG. 1;

FIG. 6 is a perspective, cutaway view of a distal end of the first member of the surgical instrument shown in FIG. 1;

FIG. 7 is an enlarged, cutaway side view of the distal end of the first member of the surgical instrument shown in FIG. 1;

FIG. 8 is an enlarged, cutaway cross-section view of the distal end of the first member of the surgical instrument shown in FIG. 1;

FIG. 9A is a front view of the distal end of the first member in a first configuration of the surgical instrument shown in FIG. 1;

FIG. 9B is a front view of the distal end of the first member in a second configuration of the surgical instrument shown in FIG. 1;

FIG. 10 is a perspective, cutaway view of the distal end of the first member including a perspective, cutaway view of the distal end of the locking pin of the surgical instrument shown in FIG. 1;

FIG. 11 is a side view in part cross-section of the surgical instrument shown in FIG. 1 and a surgical site of a body;

FIG. 12 is a side view in part cross-section of the surgical instrument and the surgical site shown in FIG. 11;

FIG. 13 is a side view in part cross-section of the surgical instrument and the surgical site shown in FIG. 11; and

FIG. 14 is a side view in part cross-section of the surgical instrument and the surgical site shown in FIG. 11.

Like reference numerals indicate similar parts throughout the figures.

### DETAILED DESCRIPTION OF THE INVENTION

The exemplary embodiments of the surgical instrument and related methods of use disclosed are discussed in terms of medical devices for the treatment of musculoskeletal disorders and more particularly, in terms of a surgical instrument and method. The surgical instrument includes a first member having an expandable head for mating with a head of a threaded member, and a locking pin that is insertable through a bore defined in the first member, and configured to move the expandable head from a first configuration to a second configuration. In the first configuration the expandable head mates loosely with the head of the threaded member and in the second configuration the expandable head mates tightly with the head of the threaded member. The surgical instrument in the second configuration enables the threaded member to be held by the surgical instrument and allow the surgical instrument to thread the threaded member into a surgical site. The surgical instrument allows a surgeon to insert a threaded member using only one hand in that the surgical instrument holds the threaded member. It is further envisioned that the surgical instrument is configured to deliver various surgical components such as, for example, bone screws, locking screws, set screws and other threaded members. The surgical instrument and method may be employed with an imaging or surgical navigation system.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical instrument may be employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, left and right, are for illustrative purposes only and can be varied within the scope of the disclosure. For example, the references “upper” and “lower” are relative and used only in the context to the other, and are not necessarily “superior” and “inferior”.

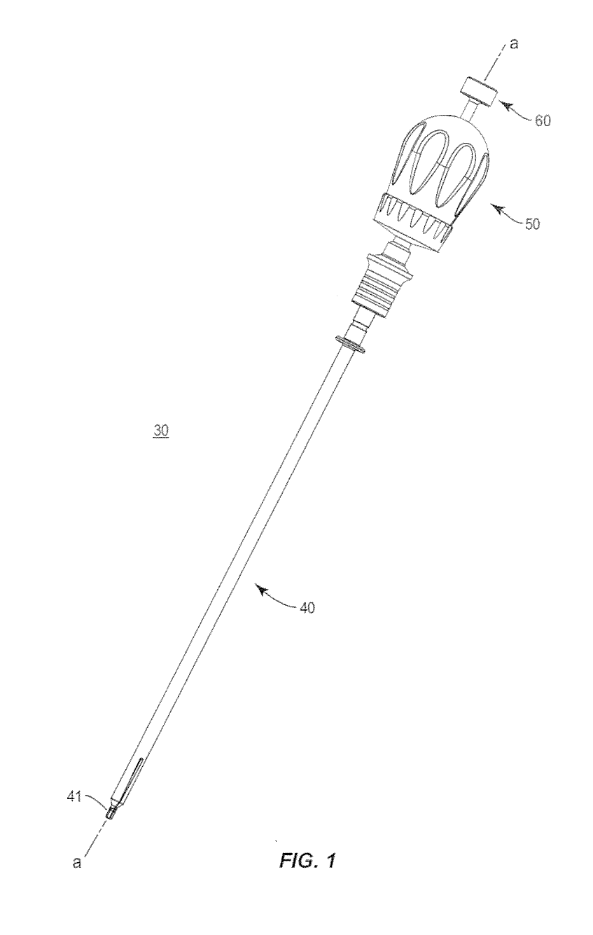

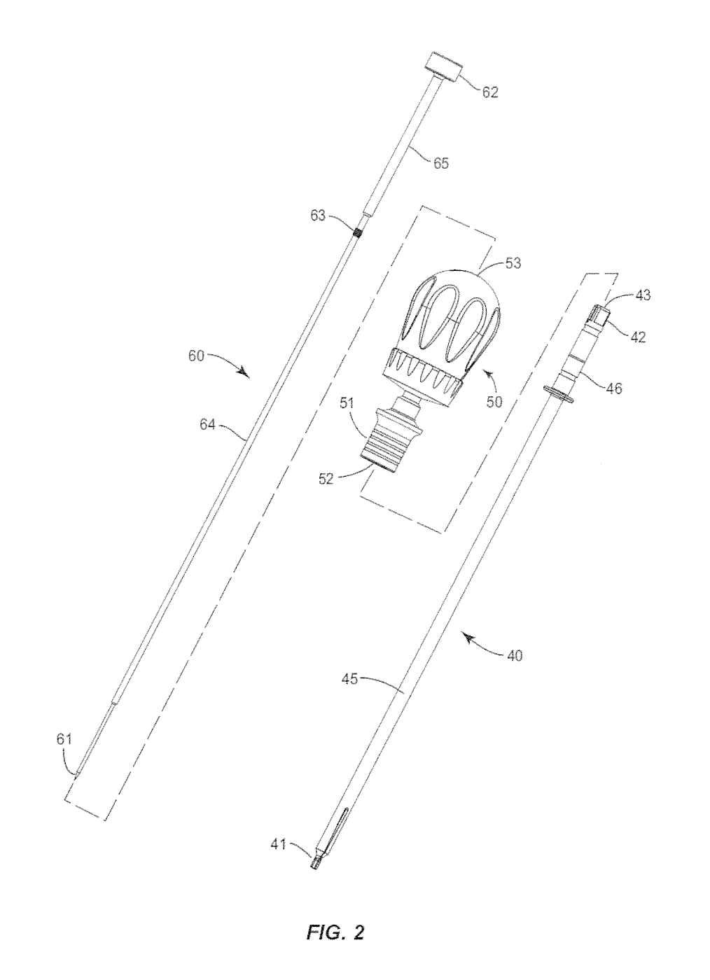

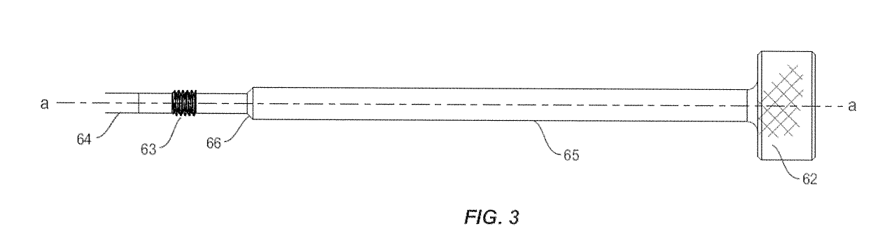

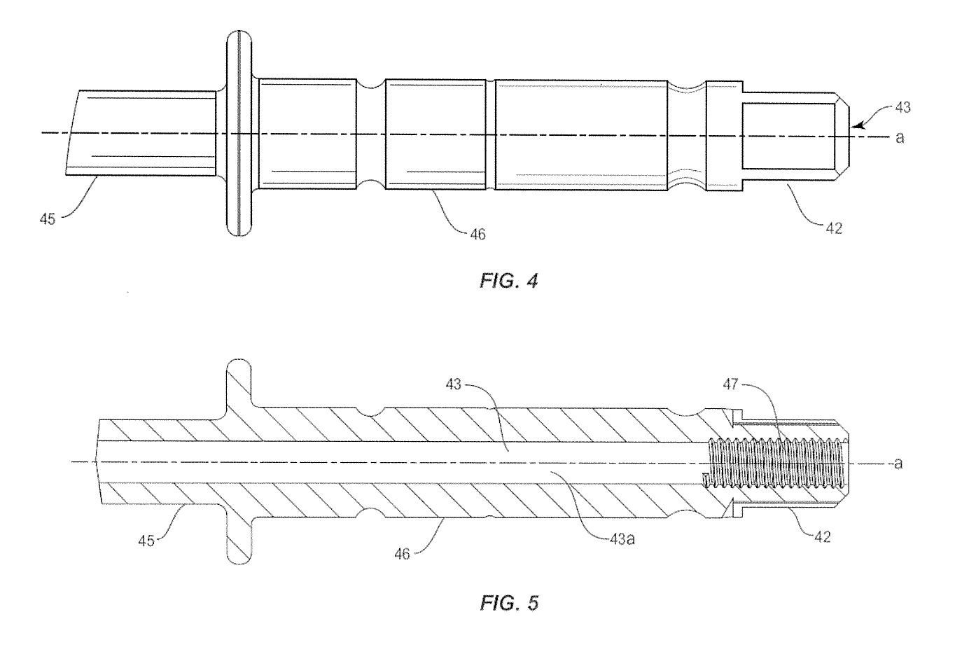

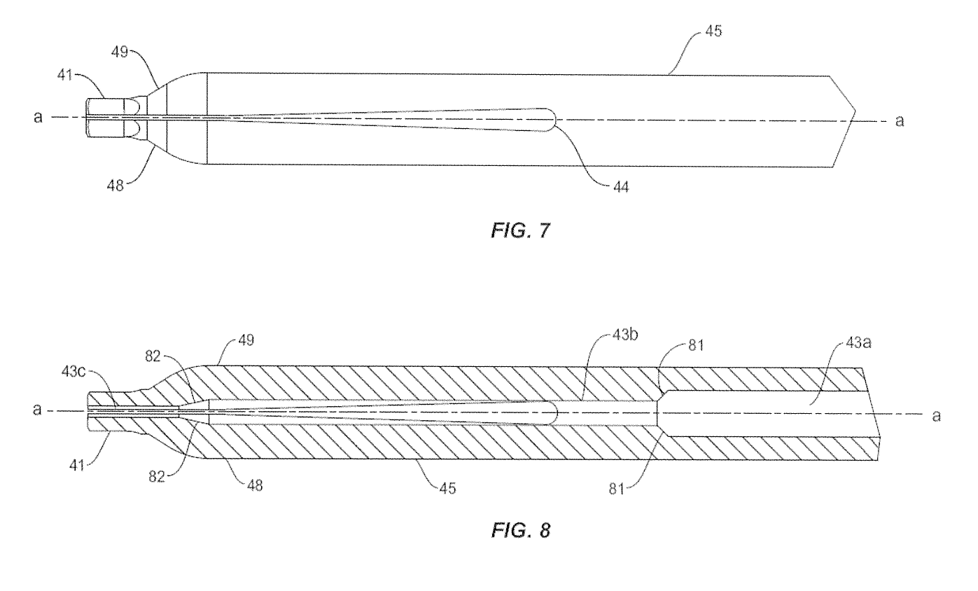

The following discussion includes a description of a surgical instrument and related methods of employing the surgical instrument in accordance with the principles of the present disclosure. Alternate embodiments are also disclosed. Reference will now be made in detail to the exemplary embodiments of the present disclosure, which are illustrated in the accompanying drawings. Turning now to FIGS. 1-10, there is illustrated components of a surgical instrument 30 in accordance with the principles of the present disclosure.

The components of surgical instrument 30 are fabricated from materials suitable for medical applications, including metals, polymers, ceramics, biocompatible materials and/or their composites, depending on the particular application and/or preference of a medical practitioner. For example, the components of surgical instrument 30, individually or collectively, and which may be monolithically formed or integrally connected, can be fabricated from materials such as stainless steel, stainless steel alloys, titanium, titanium alloys, super-elastic titanium alloys, cobalt-chrome alloys, shape memory materials, such as super-elastic metallic alloys (e.g., Nitinol, super-elastic plastic metals, such as GUM METAL® manufactured by Toyotsu Material Incorporated of Japan), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, biocompatible materials such as polymers including plastics, metals, ceramics and composites thereof, rigid polymers including polyphenylene, polyamide, polyimide, polyetherimide, polyethylene, and epoxy. Various components of surgical instrument 30 may have material composites, including the above materials, to achieve various desired characteristics such as strength, rigidity, elasticity, compliance, biomechanical performance, durability and radiolucency or imaging preference.

Surgical instrument 30 is configured to deliver a surgical screw to a surgical site at a desired location. Surgical instrument 30 includes a first member 40, a handle 50 and a locking pin 60. First member 40, handle 50 and locking pin 60 each have its longitudinal axis positioned along axis a. First member 40 terminates at a distal end at head 41. Head 41 is configured to mate with a head of a surgical screw.

Surgical screws are used in multiple surgical procedures. Some surgical screws, such as bone screws or anchors, are designed to penetrate cartilage and/or bone and serve as an anchor for other surgical devices, such as rods and pins. Other surgical screws, such as set screws, serve to fix together 2 members of a surgical device, for example, a set screw may be used to fix a rod to a bone screw. These surgical screws usually include a head socket that is matable with a driver to drive the screw. For example, certain surgical screws include a hexagonal female head socket that is matable with a hex driver to drive the screw. Other head socket and driver shapes, such as star, square, triangle, etc., are contemplated. In addition, various sizes of head socket and driver are contemplated. During insertion, a surgeon is required to hold both the surgical screw and driver, at least until the threads of the screw are engaged within a receptacle.

Surgical instrument 30 is designed such that head 41 is expandable between a first configuration and a second configuration. In the first configuration, head 41 loosely mates with a head of a surgical screw. In the second configuration, head 41 is expanded to tightly mate with the head of the surgical screw and hold the screw thereon. The surgical instrument 30 can itself hold the screw during the driving process, thus freeing a hand of the surgeon. The expansion of head 41 is performed by an interaction between first member 40 and locking pin 60, as described in further detail herein.

First member 40 includes head 41 connected to shaft 45 at a distal end thereof. A proximal end of shaft 45 terminates with handle shaft 46 and male end 42. The length of first member 40 along its longitudinal axis is defined as L1. First member 40 defines a bore 43 extending throughout first member 40 along longitudinal axis a. At a proximal end of first member 40, bore 43 includes threads 47 as shown in FIG. 5.

Although bore 43 can be of a single diameter throughout, in a preferred embodiment bore 43 is graduated from a larger diameter at proximal end of first member 40 to a smaller diameter at distal end of first member 40. FIG. 8 illustrates bore 43 having a graduating diameter of bore 43a of a first diameter d1, bore 43b of a second diameter d2, and bore 43c of a third diameter d3, where d1>d2>d3. This graduating configuration allows for greater ease when inserting locking pin 60 through bore 43, as described in further detail herein. Angled transition points 81 and 82 transition between bore 43a, 43b and 43b, 43c, respectively, and assist in the guiding of locking pin 60 through bore 43, as described herein.

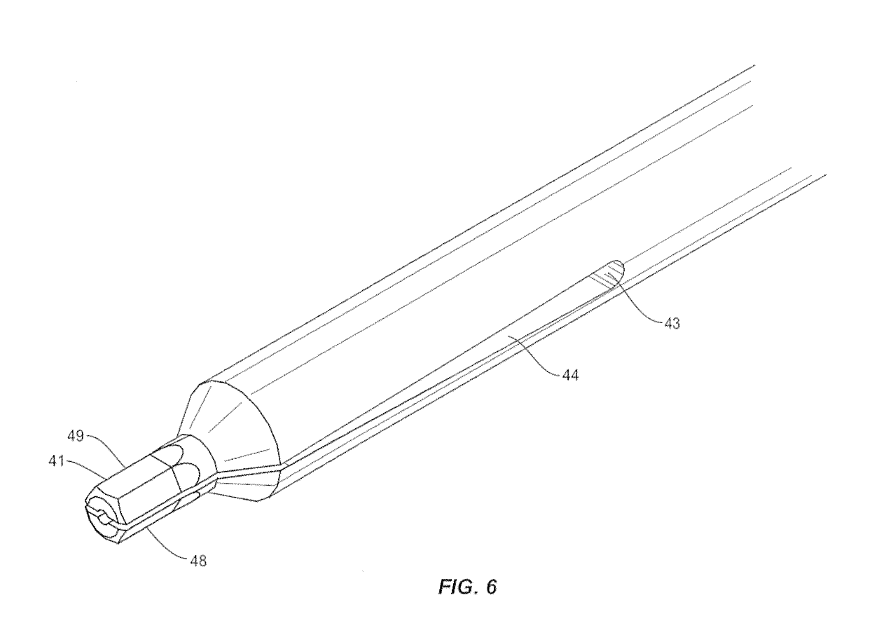

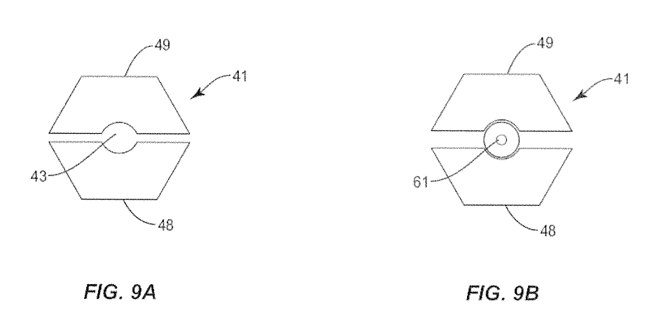

Distal end of first member 40 includes a first split end 48 and a second split end 49 that define a slit 44 extending from distal end a distance along said longitudinal axis a as shown in FIG. 6. First member 40 is configured such that slit 44 is normally biased toward a closed first configuration as shown in FIG. 6, where distal ends of first and second split ends 48, 49 are close together. Bore 43 terminates at a proximal end of slit 44, but continues as slit 44 narrows and terminates at distal end of head 41 as shown in FIG. 9A. A distance from proximal end of first member 40 to the start of bore 43c is defined as length L2.

Handle 50 includes a female end 52 positioned at distal end 51. Female end 52 is configured to mate with male end 42 of first member 40. Handle 50 also defines a bore 53 extending throughout handle 50 along longitudinal axis a. Bore 53 has a diameter d4. When handle 50 is connected to first member 40, bore 53 aligns with bore 43. Male end 42 is shown as square in cross section and mates with female end 52 also having a square cross section configuration of similar size to male end 42; other cross-section configurations are contemplated. Female end 52 mates with male end 42 in a non-rotatable manner. Handle 50 provides a gripping surface to provide a turning force to a screw being driven by surgical instrument 30. Although handle 50 is shown as a separate element from first member 40, it is contemplated that handle 50 and first member 40 can be monolithically formed as one part. It is further contemplated that handle 50 can be of different shapes and sizes, for example, a “T” shaped handle, and may have other cross-sectional geometries such as those described herein.

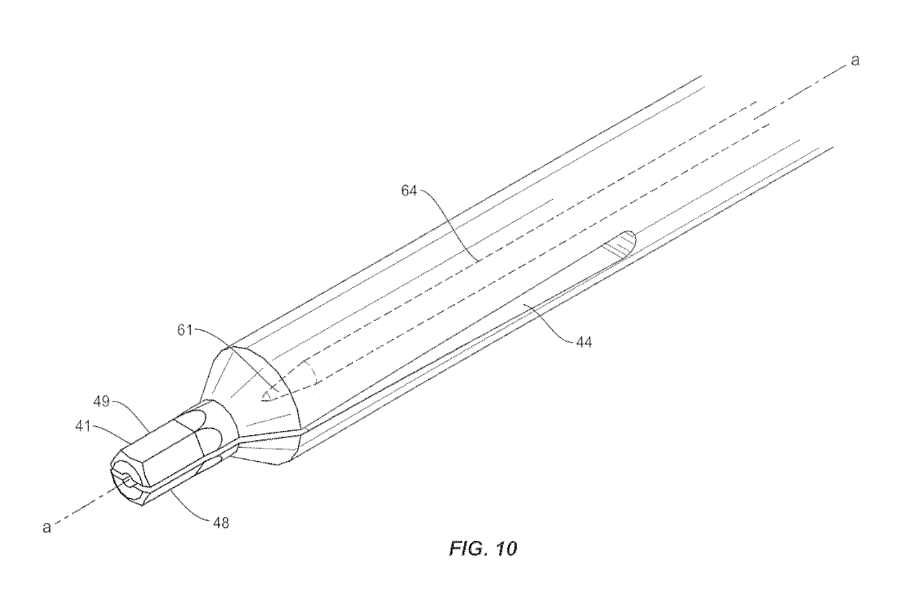

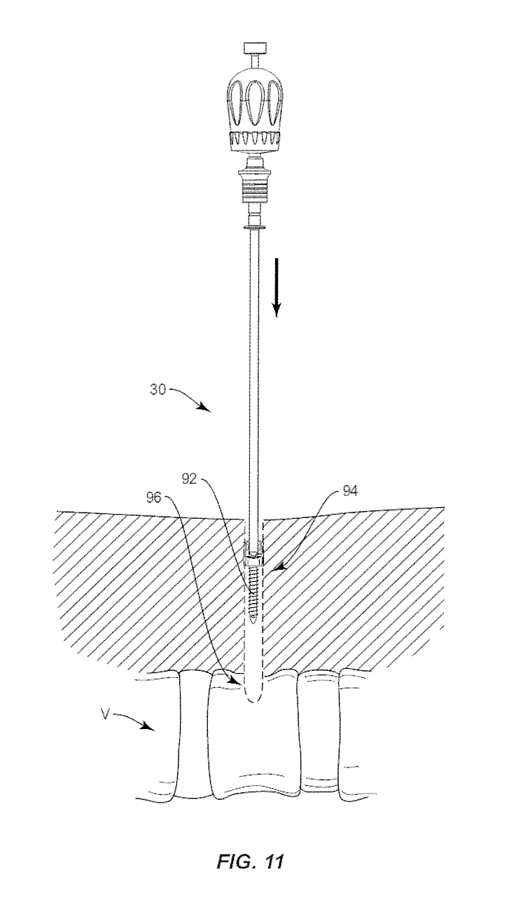

Locking pin 60 includes a shaft 64 having a distal end 61. Distal end 61 of locking pin 60 is tapered as shown in FIG. 10. A proximal end of shaft 64 includes a threaded portion 63 that is designed to mate with threads 47 of first member 40. Locking pin 60 terminates at a proximal end with knob 62 and includes an extension shaft 65 between threads 63 and knob 62. A stop 66 is positioned at the distal end of extension shaft 65. Shaft 64 defines a diameter d5 and extension shaft 65 defines a diameter d6, where d5<d6. Shaft 64 defines a length L3, and extension shaft 65 defines a length L4. A distance between threads 63 and stop 66 is defined as length L5. A distance between distal end 61 and stop 66 is defined as length L6. In addition, the diameter d6 of extension shaft 65 is less than diameter d4 of bore 53 of handle 50 to allow extension shaft 65 to pass through bore 53.

Upon assembly of surgical instrument 30, female end 52 of handle 50 is attached onto male end 42 of first member 40. As stated above, handle 50 and first member 40 may be monolithically formed. Locking pin 40 is then inserted into and through bore 53 and advanced into bore 43. Diameter d5 of shaft 64 is less than both diameter d1 and d2 of bore 43a and 43b, respectively, and less than diameter d4 of bore 53. This allows for the free sliding movement of locking pin 60 within bores 43a, 43b and 53. Locking pin 60 is advanced to through bore 43 to a point where distal end 61 engages with bore 43c as shown in FIG. 10. Diameter d3 of bore 43c is less than diameter d5 of shaft 64 when split ends 48, 49 are biased toward each other in the first and closed configuration as shown in FIG. 9A. In this first configuration, a head of a screw would loosely mate with head 41 since the size of head 41 in the first configuration is slightly less than the size of a female socket of the head of the screw.

Length L3 of shaft 64 is defined such that when distal end 61 begins to engage with bore 43c, threads 63 also begin to engage with threads 47. At this point, in order to further advance locking pin 60 through bore 43, locking pin 60 needs to be rotated, preferably by use of knob 62, to engage threads 63 and 47. As the threaded engagement of threads 63, 47 continues, distal end 61 of locking pin 60 will further advance into bore 43c forcing apart split ends 48, 49 and expanding head 41 into the second and expanded configuration as shown in FIG. 9B. In this second configuration, the head of the screw would tightly mate with head 41 since split ends 48, 49 of head 41 in the second configuration are forced open to provide a holding force on the female socket of the head of the screw. To release the screw from head 41, an opposite rotation is provided to knob 62 to withdraw the distal end 61 of locking pin 60 from bore 43c, thus allowing the bias of split ends 48, 49 to the first configuration.

If distal end 61 of locking pin 60 were to extend beyond head 41 of first member 40 when a screw is mated therewith, distal end 41 would tend to push the screw in a direction away from head 41 and possible disengage screw from head 41, even in the second configuration. Stop 66 is provided to prevent distal end 61 of locking pin 60 from extending beyond head 41 of first member 40. This is accomplished by the following configurations.

First, diameter d6 of extension shaft 65, that is of stop 66, is greater than diameter d1 of bore 43a, thus preventing extension shaft 65 of locking pin 60 from entering into bore 43a of first member 40. It is contemplated that as long as stop 66 has a diameter greater than diameter d1 of bore 43a, even though the diameter of extension shaft 65 might be less than diameter d1 of bore 43a, stop 66 would prevent locking pin 60 from entering bore 43 passed stop 66.

Second, length L6 of locking pin 60 from distal end 61 to stop 66 is less than or equal to length L1 of first member 40. As stated above, length L6 of locking pin 60 from distal end 61 to stop 66 must be greater than length L2 of first member 40 from proximal end to bore 43c to allow distal end 61 to expand split ends 48, 49 into the second configuration.

In assembly, operation and use, surgical instrument 30 is assembled as described above and employed with a minimally invasive surgical procedure with a section of a spine of a patient. It is envisioned that surgical instrument 30 may be employed for performing spinal surgeries, such as, for example, discectomy, laminectomy, fusion, laminotomy, laminectomy, nerve root retraction, foramenotomy, facetectomy, decompression, spinal nucleus or disc replacement, bone graft and implantation of prosthetics including plates, rods, and bone engaging fasteners used in any existing surgical method or technique including open surgery, mini-open surgery, minimally invasive surgery and percutaneous surgical implantation.

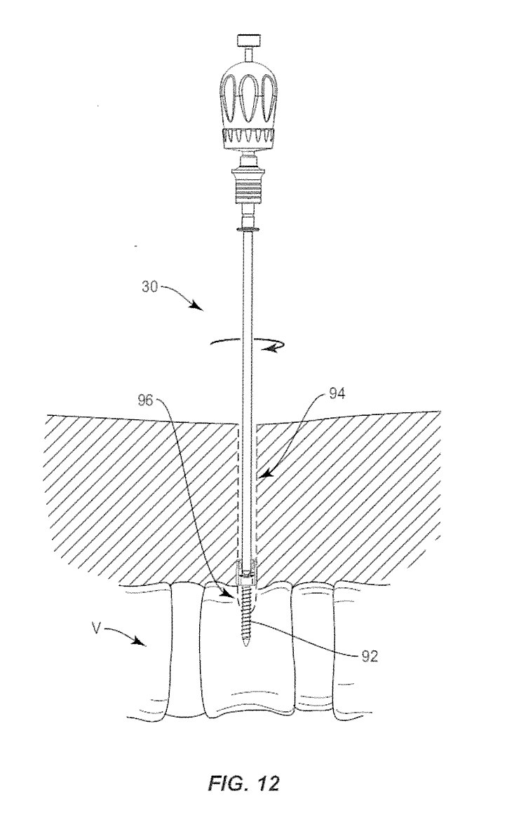

Turning now to FIGS. 11-14, there is illustrated methods of using surgical instrument 30 in accordance with the principles of the present disclosure. For example, surgical instrument 30 is employed with a percutaneous approach for treating the spine section. A cannula, mini-open retractor, tube, a sleeve for slidable support of surgical instrument 30 provides a protected passageway for surgical instrument 30 such that surgical instrument 30 in the second configuration with a screw attached thereto can be advanced to the surgical site. A medical practitioner will make an incision in the skin of a patient's body to create a protected passageway 94 over and in approximate alignment with vertebrae V at the surgical site. A sleeve or other dilator may be employed to separate the muscles and tissues to create passageway 94 through which the surgery may be performed. Passageway 94 allows for the insertion and use of surgical instrument 30.

Passageway 94 is created and extends from the incision to adjacent vertebrae V. A bore 96 is pre-drilled in tissue, such as, for example, bone of vertebrae V prior to insertion of bone screw 92. Bore 96 is configured to receive bone screw 92. It is envisioned that passageway 94 is disposed at various angular orientations relative to vertebrae V. It is further envisioned that passageway 94 may extend outside a patient's body using various instruments as described herein.

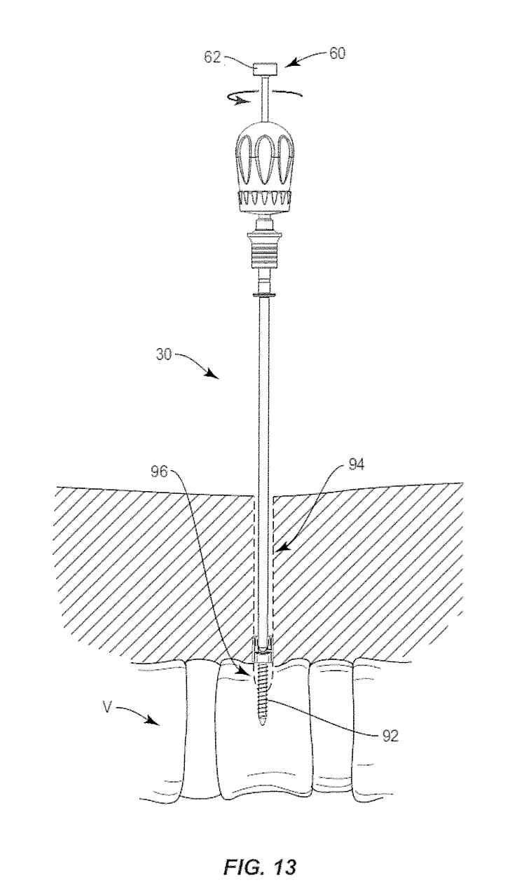

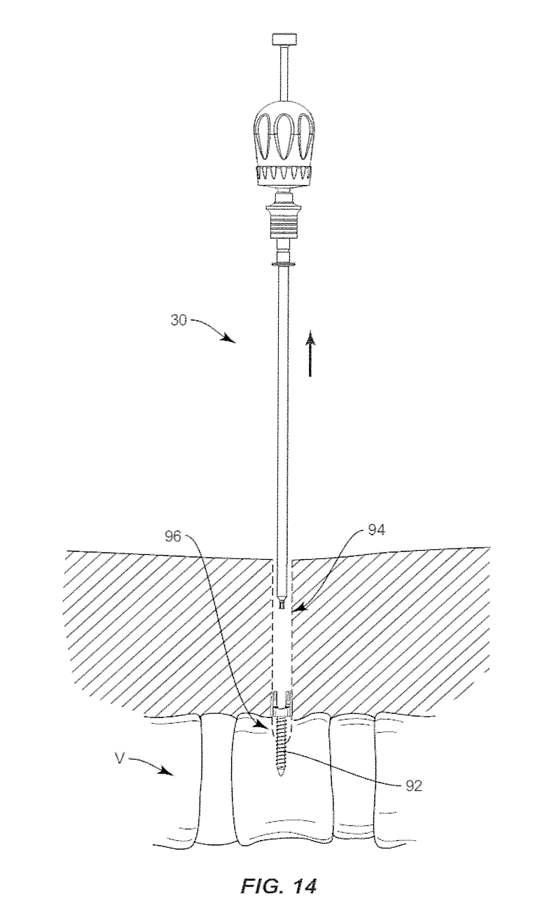

Bone screw 92 is mated with head 41 in the first configuration, and affixed thereto via expansion of head 41 into the second configuration as described above. Surgical instrument 30 with bone screw 92 affixed thereto is inserted within passageway 94, as shown in FIG. 11. Surgical instrument 30 with bone screw 92 affixed thereto is advanced through passageway 94 until bone screw 92 contacts vertebrae V at bore 96. At this point, bone screw is driven into vertebrae V by turning surgical instrument 30 with bone screw 92 affixed thereto as shown in FIG. 12. When bone screw 92 is properly positioned in vertebrae V, head 41 of surgical instrument 30 is released from bone screw 92 by rotating locking pin 60 via knob 62 to back distal end 61 out of bore 43c, thus allowing split ends 48, 49 to bias toward the first configuration as shown in FIG. 13. Finally, surgical instrument 30 is removed from passageway 94 leaving bone screw 92 in vertebrae V as shown in FIG. 14.

It is envisioned that the use of microsurgical and image guided technologies may be employed to access, view and repair spinal deterioration or damage, with the aid of surgical instrument 30. Upon completion of the procedure, surgical instrument 30 is removed and the incision is closed. It is contemplated that a surgical procedure employing surgical instrument 30 may be used with various surgical components, such as, for example, implants, surgical tools and surgical instruments, such as, rasps, curettes, nerve root retractors, tissue retractors, forceps, cutter, drills, scrapers, reamers, separators, rongeurs, taps, cauterization instruments, irrigation and/or aspiration instruments, illumination instruments and/or inserter instruments.

In assembly, operation and use, surgical instrument 30 is assembled as described above and employed with other minimally invasive surgical procedure with a section of a spine of a patient. It is further envisioned that surgical instrument 30 may be employed for performing spinal surgeries, such as, for example, inserting a facet screw through a facet joint of adjoining vertebrae. The facet screw is inserted through the facet joint to fix the facets of the adjoining vertebrae to each other. The facet screws can be used to treat spinal trauma or supplement other types of vertebral fusions.

It is contemplated that locking pin 60 can define a bore (not shown) therethrough along its longitudinal axis. This bore in locking pin 60 is configured such that surgical instrument 30 can be used in surgical procedures that include guide wire applications. For example, bone screws and set screws for use in certain surgical procedures can include guide wire bores to guide them into position in the patient's body. With the bore through locking pin 60, a guide wire can be used to guide assembled surgical instrument 30 with bone screw or set screw attached to head 41 to a proper position in the patient's body.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument, comprising:
a first member extending along a longitudinal axis having a first end configured to mate with a head of a threaded member, said first end having a first configuration such that said first end loosely mates with said head of said threaded member and a second configuration such that said first end tightly mates with said head of said threaded member; and
a second member configured to expand said head from said first configuration to said second configuration.

2. The surgical instrument of claim 1, wherein said second member is a locking pin insertable into a bore extending through said first member along said longitudinal axis.

3. The surgical instrument of claim 2, wherein said first end of said first member defines a slit extending from said first end a distance along said longitudinal axis.

4. The surgical instrument of claim 3, wherein said first end is biased toward said first configuration along said slit.

5. The surgical instrument of claim 4, wherein said first end is expanded toward said second configuration along said slit as said locking pin is advanced through said bore, along said slit and into said first end.

6. The surgical instrument of claim 2, further comprising a handle positioned at a second end opposite said first end of said first member, wherein said handle defines a bore aligned with said bore of said first member, and wherein said locking pin is insertable through said bore of said handle to continue through said bore of said first member.

7. The surgical instrument of claim 6, wherein said handle is monolithically formed with said first member.

8. The surgical instrument of claim 2, wherein said locking pin defines a locking pin bore therethrough along said longitudinal axis, said locking pin bore configured to accommodate a guide wire.

9. The surgical instrument of claim 2, wherein locking pin is threadingly engageable with said first member to advance said locking pin through at least a portion of said bore.

10. A surgical instrument, comprising:
a first member extending along a longitudinal axis having a first end configured to mate with a head of a threaded member, said first end having a first configuration such that said first end loosely mates with said head of said threaded member and a second configuration such that said first end tightly mates with said head of said threaded member;
a locking pin insertable into a bore extending through the first member along said longitudinal axis, configured to expand said head from said first configuration to said second configuration, threadingly engageable with said first member to advance said locking pin through at least a portion of said bore; and
a handle positioned at a second end opposite said first end of said first member, wherein said handle defines a bore aligned with said bore of said first member, and wherein said locking pin is insertable through said bore of said handle to continue through said bore of said first member.

11. The surgical instrument of claim 10, wherein said first end of said first member defines a slit extending from said first end a distance along said longitudinal axis.

12. The surgical instrument of claim 11, wherein said first end is biased toward said first configuration along said slit.

13. The surgical instrument of claim 12, wherein said first end is expanded toward said second configuration along said slit as said locking pin is advanced through said bore, along said slit and into said first end.

14. The surgical instrument of claim 10, wherein said locking pin defines a locking pin bore therethrough along said longitudinal axis, said locking pin bore configured to accommodate a guide wire.

15. A method of providing a threaded member during a surgical procedure, comprising the steps of:
providing a surgical instrument having a first configuration wherein a head of the surgical instrument loosely mates with a head of the threaded member and a second configuration wherein the head of the surgical instrument tightly mates with the head of the threaded member;
providing the threaded member;
mating the threaded member with the head of the surgical instrument;
expanding the surgical instrument from the first configuration to the second configuration to provide a holding force to the threaded member;
positioning the surgical instrument and the threaded member in a patient;
driving the threaded member into a desired position;
collapsing the surgical instrument from the second configuration to the first configuration;
removing the surgical instrument from the threaded member; and
removing the surgical instrument from the patient.

16. The method of claim 15, wherein the step of expanding the surgical instrument, comprises:
providing a locking pin;
inserting the locking pin into a bore extending through the surgical instrument along a longitudinal axis thereof; and
advancing the locking pin through the surgical instrument and into the head of the surgical instrument to expand the head to tightly mate with the threaded member.

17. The method of claim 16, wherein the step of collapsing the surgical instrument, comprises withdrawing the locking pin from the head of the surgical instrument to collapse the head to loosely mate with threaded member.

18. The method of claim 17, wherein the locking pin is threadingly advanced and withdrawn through at least a portion of the surgical instrument.

19. The method of claim 18, wherein the step of positioning the surgical instrument and the threaded member in the patient, comprises:
providing a guide wire into the patient; and
guiding the surgical instrument, the locking pin and the threaded member over the guide wire and into the patient.

20. The method of claim 19, wherein the step of driving the threaded member, comprises:
providing a handle;
attaching the handle to the surgical instrument; and
turning the handle to drive the threaded member.
